SEQUENCE LISTING (16 sequences)

SEQ ID NO: 1 - RNA, 23 nt, Homo sapiens
uaagugcuuccauguuuugguga

SEQ ID NO: 2 - DNA, 34 bp, Artificial Sequence
  Synthetic oligonucleotide primer
cgaggtattcgcactggatacgactcaccaaaac

SEQ ID NO: 3 - DNA, 54 bp, Artificial Sequence
  Synthetic oligonucleotide primer
gtcgtatccagtgcagggtccgaggtattcgcactggatacgactcaccaaaac

SEQ ID NO: 4 - DNA, 24 bp, Artificial Sequence
  Synthetic oligonucleotide
gtcgtatccagtgcgaatacctcg

SEQ ID NO: 5 - DNA, 17 bp, Artificial Sequence
  Synthetic oligonucleotide primer
taagtgcttccatgttt

SEQ ID NO: 6 - DNA, 13 bp, Artificial Sequence
  Synthetic oligonucleotide primer
cgaggtattcgca

SEQ ID NO: 7 - DNA, 13 bp, Artificial Sequence
  Synthetic oligonucleotide probe
atacgactcacca

SEQ ID NO: 8 - DNA, 21 bp, Artificial Sequence
  Synthetic oligonucleotide
gtcgtatccagtgcgaatacc

SEQ ID NO: 9 - DNA, 18 bp, Artificial Sequence
  Synthetic oligonucleotide
gtcgtatccagtgcgaat

SEQ ID NO: 10 - DNA, 24 bp, Artificial Sequence
  Synthetic oligonucleotide
gtcgtatccattgctaatacctcg

SEQ ID NO: 11 - DNA, 24 bp, Artificial Sequence
  Synthetic oligonucleotide
gtcgtatccattactaatacctcg

SEQ ID NO: 12 - DNA, 24 bp, Artificial Sequence
  Synthetic oligonucleotide
gttgtgttcggtgtggatgctttg

SEQ ID NO: 13 - DNA, 24 bp, Artificial Sequence
  Synthetic oligonucleotide
gttgtgtttggtgtgggtgttttg

SEQ ID NO: 14 - DNA, 37 bp, Artificial Sequence
  Synthetic oligonucleotide primer
gtccgaggtattcgcactggatacgactcaccaaaac

SEQ ID NO: 15 - DNA, 27 bp, Artificial Sequence
  Synthetic oligonucleotide
gtcgtatccagtgcgaatacctcggac

SEQ ID NO: 16 - DNA, 17 bp, Artificial Sequence
  Synthetic oligonucleotide primer
ccgaggtattcgcactg

(19) United States
(12) Patent Application Publication
(10) Pub. No.: US 2020/0010829 A1
(43) Pub. Date: Jan. 9, 2020

(54) METHOD FOR SYNTHESIZING cDNA

(71) Applicant: SYSMEX CORPORATION, Kobe-shi (JP)

(72) Inventors: Kanako MASUMOTO, Kobe-shi (JP); Yuki AIHARA, Kobe-shi (JP)

(73) Assignee: SYSMEX CORPORATION, Kobe-shi (JP)

(21) Appl. No.: 16/425,175

(22) Filed: May 29, 2019

(30) Foreign Application Priority Data
May 30, 2018 (JP) 2018-103590

(51) Int. Cl. C12N 15/10; C12Q 1/6876

(52) U.S. Cl. CPC C12N 15/1096; C12Q 1/6876

(57) ABSTRACT

Disclosed is a method for synthesizing cDNA by mixing a target RNA, a first oligonucleotide molecule, a second oligonucleotide molecule, and a reverse transcriptase, using the target RNA as a template, the first oligonucleotide molecule having a first region that hybridizes to the target RNA at 3′ end, and having a second region that hybridizes to the second oligonucleotide molecule on 5′ side of the first region.

### CROSS REFERENCE TO RELATED APPLICATIONS

This application claims priority from prior Japanese Patent Application No. 2018-103590 filed on May 30, 2018, entitled “Method for measuring synthesizing cDNA, method for detecting target RNA and reagent kit”, the entire contents of which are incorporated herein by reference.

### TECHNICAL FIELD

The present invention relates to a method for synthesizing cDNA.

### BACKGROUND

In recent years, it has become apparent that small noncoding RNAs represented by microRNAs (miRNAs) play important roles in various biological processes such as development, differentiation, cell proliferation, and apoptosis. Detecting and quantifying such functional, small RNAs are very important in understanding biological phenomena. Generally, for detection of RNA, a method for synthesizing cDNA from RNA by reverse transcription reaction and amplifying and detecting the cDNA by PCR method is often used. However, since the nucleotide length of miRNA is as short as about 20 bases, it is difficult to design a primer that hybridizes to both ends of cDNA obtained by the reverse transcription reaction.

As a method for synthesizing and amplifying cDNA from miRNA, for example, the methods described in EP 1 851 336 B and U.S. Pat. No. 7,575,863 are known. EP 1 851 336 B discloses that a reverse transcription reaction of miRNA is performed using a primer having a region that does not hybridize to miRNA on 5′ side to synthesize cDNA longer than the miRNA (see FIG. 1 of EP 1 851 336 B). U.S. Pat. No. 7,575,863 discloses that a reverse transcription reaction of miRNA is performed using a stem-loop structure primer having a region that hybridizes to the miRNA as an overhang portion (see FIG. 4 of U.S. Pat. No. 7,575,863). In the method of U.S. Pat. No. 7,575,863, after the reverse transcription reaction, a stem portion of the primer is dissociated to obtain cDNA longer than the miRNA. The methods of EP 1 851 336 B and U.S. Pat. No. 7,575,863 facilitate amplification of cDNA by PCR using the obtained cDNA as a template.

The present inventors have found that the methods of EP 1 851 336 B and U.S. Pat. No. 7,575,863 leave room for improvement in sensitivity and specificity in the synthesis and amplification of cDNA from small RNAs.

### SUMMARY

The scope of the present invention is defined solely by the appended claims, and is not affected to any degree by the statements within this summary.

Accordingly, a first aspect of the present invention provides a method for synthesizing cDNA by mixing a target RNA, a first oligonucleotide molecule, a second oligonucleotide molecule, and a reverse transcriptase, using the target RNA as a template, the first oligonucleotide molecule comprising a first region that hybridizes to the target RNA at 3′ end, and comprising a second region that hybridizes to the second oligonucleotide molecule on 5′ side of the first region.

A second aspect of the present invention provides a method for synthesizing cDNA, comprising

contacting a template target RNA, a first oligonucleotide molecule, a second oligonucleotide molecule, and a reverse transcriptase, the first oligonucleotide molecule comprising a first region that hybridizes to the target RNA at 3′ end, and comprising a second region that hybridizes to the second oligonucleotide molecule on 5′ side of the first region, and

conducting extension reaction from the 3′ end of the first region of the first oligonucleotide molecule to synthesize cDNA.

A third aspect of the present invention provides a method for synthesizing a cDNA, comprising

conducting a reverse transcription reaction of a target RNA in a sample with a first oligonucleotide molecule and a second oligonucleotide molecule to synthesize the cDNA, the first oligonucleotide molecule comprising a first region that hybridizes to the target RNA at 3′ end, and comprising a second region that hybridizes to the second oligonucleotide molecule on 5′ side of the first region, and

amplifying and detecting the cDNA.

### BRIEF DESCRIPTION OF THE DRAWINGS

FIG. 1 is a schematic diagram showing a reaction principle in synthesis and amplification of cDNA by a synthesis method of a present embodiment;

FIG. 2A is a view showing an example of an appearance of a reagent kit according to the present embodiment;

FIG. 2B is a view showing an example of an appearance of a reagent kit according to the present embodiment;

FIG. 2C is a view showing an example of an appearance of a reagent kit according to the present embodiment;

FIG. 2D is a view showing an example of an appearance of a reagent kit according to the present embodiment;

FIG. 3 is an amplification plot when cDNA synthesized by a method of EP 1 851 336 B is amplified by real-time PCR;

FIG. 4 is an amplification plot when cDNA synthesized by a method of U.S. Pat. No. 7,575,863 is amplified by real-time PCR;

FIG. 5 is an amplification plot when cDNA synthesized by a method for synthesizing cDNA of the present embodiment is amplified by real-time PCR;

FIG. 6A is an amplification plot when cDNA synthesized by the method for synthesizing cDNA of the present embodiment using a second oligonucleotide molecule having a base sequence fully complementary to a base sequence of a second region of a first oligonucleotide molecule is amplified by real-time PCR;

FIG. 6B is an amplification plot when cDNA synthesized by the method for synthesizing cDNA of the present embodiment using a second oligonucleotide molecule having a base sequence containing a base that is not complementary to a base sequence of a second region of a first oligonucleotide molecule is amplified by real-time PCR;

FIG. 6C is an amplification plot when cDNA synthesized by the method for synthesizing cDNA of the present embodiment using a second oligonucleotide molecule having a base sequence containing a base that is not complementary to a base sequence of a second region of a first oligonucleotide molecule is amplified by real-time PCR;

FIG. 6D is an amplification plot when cDNA synthesized by the method for synthesizing cDNA of the present embodiment using a second oligonucleotide molecule having a base sequence containing a base that is not complementary to a base sequence of a second region of a first oligonucleotide molecule is amplified by real-time PCR;

FIG. 6E is an amplification plot when cDNA synthesized by the method for synthesizing cDNA of the present embodiment using a second oligonucleotide molecule having a base sequence containing a base that is not complementary to a base sequence of a second region of a first oligonucleotide molecule is amplified by real-time PCR;

FIG. 7A is an amplification plot when cDNA synthesized by the method for synthesizing cDNA of the present embodiment using a second oligonucleotide molecule into which a cap structure is introduced at 3′ end is amplified by real-time PCR; and

FIG. 7B is an amplification plot when cDNA synthesized by the method for synthesizing cDNA of the present embodiment using a second oligonucleotide molecule into which a cap structure is introduced at 3′ end is amplified by real-time PCR.

### DETAILED DESCRIPTION OF THE PREFERRED EMBODIMENTS

As used herein, the expression “hybridize” refers to that a whole or part of a predetermined oligonucleotide (or polynucleotide) forms a double strand via a hydrogen bond with a whole or part of another oligonucleotide (or polynucleotide) under stringent conditions. The “stringent condition” may be a condition commonly used by a person skilled in the art when performing hybridization of oligonucleotides (or polynucleotides) Examples thereof include conditions in which one oligonucleotide molecule can specifically hybridize to the other oligonucleotide molecule when there is at least 50%, preferably at least 75%, and more preferably at least 90% sequence identity between the two oligonucleotide molecules. Stringency of hybridization is known to be a function of temperature, salt concentration, nucleotide length and GC content of the oligonucleotide and concentration of a chaotropic agent contained in a hybridization buffer. As the stringent conditions, conditions described in, for example, Sambrook, J. et al., 1998, Molecular Cloning: A Laboratory Manual (2nd edition), Cold Spring Harbor Laboratory Press, New York.

As used herein, the term “base sequence fully complementary” refers to a base sequence that forms complementary base pairs of Watson-Crick model with all bases in the base sequence of a whole or part of a predetermined oligonucleotide (or polynucleotide). As used herein, the term “mismatch site” refers to a site in which complementary base pairs of Watson-Crick model cannot be formed because a base that is not complementary to a predetermined base in a base sequence of one oligonucleotide molecule (or polynucleotide molecule) is present at a corresponding position in a base sequence of the other oligonucleotide molecule (or polynucleotide molecule) when the two oligonucleotide molecules (or polynucleotide molecules) are hybridized. The meaning of the term “base sequence” equals to “nucleotide sequence.”

As used herein, the term “reaction system” means a limited environment in which components necessary for reverse transcription reaction and/or nucleic acid amplification reaction are present and the reaction occurs. Examples of a reaction system for reverse transcription reaction include tiny droplets such as a reaction solution or emulsion stored in a container such as a tube, containing a target RNA, a first oligonucleotide molecule, a second oligonucleotide molecule, and a reverse transcriptase. The term “reverse transcriptase” is synonymous with “RNA-dependent DNA polymerase”.

[1. Method for Synthesizing cDNA]

In the method for synthesizing cDNA of the present embodiment (hereinafter, also referred to as “synthesis method”), cDNA is synthesized by mixing a target RNA, a first oligonucleotide molecule, a second oligonucleotide molecule, and a reverse transcriptase, using the target RNA as a template.

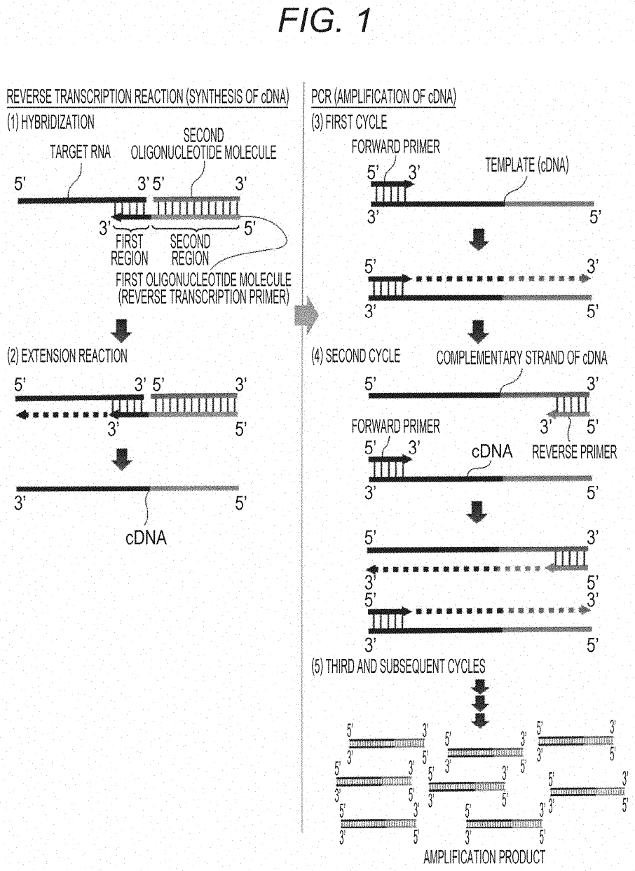

An example of a desirable reaction in the synthesis method of the present embodiment will be described with reference to FIG. 1. In this synthesis method, the first oligonucleotide molecule functions as a reverse transcription primer (hereinafter also referred to as “RT primer”). The first oligonucleotide molecule has a first region that hybridizes to the target RNA at 3′ end, and has a second region that hybridizes to the second oligonucleotide molecule on 5′ side of the first region. When the target RNA, the first oligonucleotide molecule, the second oligonucleotide molecule, and the reverse transcriptase are mixed, as shown in the left side (1) of FIG. 1, the first region of the first oligonucleotide molecule hybridizes to the target RNA, and the second region hybridizes to the second oligonucleotide molecule. In the example of FIG. 1, the first region has a base sequence fully complementary to the base sequence of the portion containing the 3′ end of the target RNA. In this example, the second oligonucleotide molecule has the same nucleotide length as the second region, and has a base sequence fully complementary to the base sequence of the second region.

By placing the above mixture under temperature conditions suitable for reverse transcription reactions, as shown in the left side (2) of FIG. 1, the 3′ end of the first oligonucleotide molecule hybridized with the target RNA and the second oligonucleotide molecule is extended to synthesize cDNA, by action of reverse transcriptase. Although cDNA is strictly a part of a complementary strand of target RNA, cDNA as used herein refers to an oligonucleotide containing not only the complementary strand of target RNA but also the second region. That is, as used herein, cDNA refers to a first oligonucleotide molecule extended using a target RNA as a template. While the synthesized cDNA may hybridize to the target RNA and/or the second oligonucleotide molecule, the target RNA and the second oligonucleotide molecule can be easily removed by heat denaturation or the like.

As shown in FIG. 1, since the second region of the first oligonucleotide molecule is a region which does not hybridize to the target RNA, cDNA longer than the target RNA can be obtained. This facilitates design of primers used for cDNA amplification to be performed later. Although the synthesis method of the present embodiment is not limited by theory or action mechanism, it is presumed that extending the 3′ end of the first oligonucleotide molecule in a state in which a double strand of the second region of the first oligonucleotide molecule and the second oligonucleotide molecule is formed contributes to improvement in accuracy and specificity of cDNA synthesis and subsequent cDNA amplification.

In the synthesis method of the present embodiment, the obtained cDNA may be amplified by a DNA amplification method such as PCR method. In this case, a mixture after reverse transcription reaction containing cDNA can be used as a template as it is. In the example of FIG. 1, cDNA is amplified by normal PCR method using a primer that hybridizes to cDNA (hereinafter referred to as “forward primer”), a primer that hybridizes to a complementary strand of the cDNA (hereinafter referred to as “reverse primer”) and a polymerase. In a first cycle of PCR, the forward primer hybridizes to cDNA as shown in the right side (3) of FIG. 1. Then, the forward primer is extended by action of polymerase to synthesize a complementary strand of the cDNA.

In a second cycle, the forward primer hybridizes to cDNA and the reverse primer hybridizes to a complementary strand of the cDNA, as shown in the right side (4) of FIG. 1. Then, each primer is extended by the action of polymerase to obtain double stranded DNA. When the mixture after reverse transcription reaction is used as it is for PCR, in the example of FIG. 1, the reverse primer can also hybridize to the second oligonucleotide molecule remaining in the mixture. However, when a sufficiently larger amount of reverse primer than the second oligonucleotide molecule is used, it has little effect on the amplification reaction. In the third and subsequent cycles, as shown in the right side (5) of FIG. 1, amplification products are exponentially obtained using double-stranded DNA as a template.

Hereinafter, the components used for the synthesis method of the present embodiment will be described.

The target RNA is not particularly limited, and can be appropriately selected from any RNA for which synthesis of cDNA is desired. In the present embodiment, in order to prepare a first oligonucleotide molecule as an RT primer, the target RNA is preferably RNA whose base sequence is known. Information on the base sequences of RNA can be obtained from databases known in the art, for example, GenBank (http://www.ncbi.nlm.nih.gov/genbank/), miRBase (http://www.mirbase.org/search.shtml), and the like.

The synthesis method of the present embodiment is particularly suitable for cDNA synthesis from small RNAs. Small RNA refers to non-coding RNA with a nucleotide length of less than 200. Examples of the small RNA include miRNA, small nuclear RNA (snRNA), small nucleolar RNA (snoRNA), piwi-interacting RNA (piRNA), primary miRNA (pri-miRNA), precursor miRNA (pre-miRNA), short interfering RNA (siRN), short hairpin RNA (shRNA), and the like. In a preferred embodiment, the target RNA is a single stranded RNA having a nucleotide length of 15 or more and less than 200.

Although the source of the target RNA is not particularly limited, it may be RNA obtained from an organism or a biological sample, or may be artificially synthesized RNA. The organism is not particularly limited as long as it has the target RNA, and may be eukaryote or prokaryote. Preferred organisms are mammals including humans. Examples of the biological sample include organs, tissues, cells, body fluids and the like collected from an organism. Examples of the body fluid include blood, plasma, serum, lymph, saliva, urine, and the like. The biological sample may be cultured cells, cultures of microorganisms such as bacteria and viruses, culture supernatants, lysates, extracts, and the like. In recent years, it is known that exosomes contain a large amount of small RNAs such as miRNA. In the present embodiment, the biological sample may be exosomes isolated from a body fluid or the like of an organism.

A method for extracting RNA from the biological sample is publicly known per se. For example, when the biological sample is a cell or a tissue, extraction of RNA can be performed as follows. First, the biological sample is mixed with a solubilizing solution containing guanidine thiocyanate and a surfactant. Physical treatment (stirring, homogenizing, ultrasonic disruption, etc.) is performed on the resulting mixed solution to release RNA contained in the biological sample into the mixed solution. Thus, RNA can be extracted from the biological sample. The extracted RNA may be purified. For example, RNA can be purified by centrifuging a mixed solution containing RNA to collect a supernatant, and extracting the supernatant with phenol/chloroform. Extraction and purification of RNA from the biological sample may be performed using a commercially available RNA extraction kit.

The first oligonucleotide molecule is an oligonucleotide molecule having a first region at the 3′ end and a second region on the 5′ side of the first region. As described above, the first oligonucleotide molecule plays a role of RT primer in the synthesis method of the present embodiment. The first region is a region that hybridizes to the target RNA, and the second region is a region that hybridizes to the second oligonucleotide molecule. In the present embodiment, the first oligonucleotide molecule may have another region in addition to the first region and the second region as long as extension of the 3′ end by reverse transcription reaction is possible. In a preferred embodiment, the first oligonucleotide molecule includes the first region and the second region. The first region and the second region will be described below.

The first region is a region containing the 3′ end of the first oligonucleotide molecule. The first region of the first oligonucleotide molecule hybridizes to the target RNA, and functions as an RT primer by having the 3′ end extended. The nucleotide length of the first region is not particularly limited as long as it can maintain hybridization with the target RNA during the reverse transcription reaction. The first region has, for example, a nucleotide length of 3 or more and 15 or less, preferably 4 or more and 12 or less, and more preferably 6 or more and 10 or less. The base sequence of the first region is not particularly limited as long as it is a base sequence capable of hybridizing to the target RNA. Preferably, the base sequence of the first region is a base sequence fully complementary to a base sequence of a part of the target RNA. The portion to which the first region of the target RNA hybridizes is not particularly limited, and is preferably a portion containing the 3′ end of the target RNA.

The second region is a region located on the 5′ side of the first region. The second region may be a region containing 5′ end of the first oligonucleotide molecule. As described above, the second region of the first oligonucleotide molecule hybridizes to the second oligonucleotide molecule. In the present embodiment, a whole of the second region may hybridize to the second oligonucleotide molecule. Alternatively, a part of the second region may hybridize to the second oligonucleotide molecule. Therefore, a whole or part of the second region has a base sequence capable of hybridizing to the second oligonucleotide molecule. Preferably, the base sequence of a whole or part of the second region is a base sequence fully complementary to an entire base sequence of the second oligonucleotide molecule. The nucleotide length of the second region is not particularly limited, and is, for example, a nucleotide length of 10 or more and 40 or less, preferably 17 or more and 40 or less, and more preferably 20 or more and 40 or less.

In the synthesis method of the present embodiment, the second region is an additional sequence that imparts a length to a complementary strand of target RNA synthesized by the reverse transcription reaction. Therefore, the second region is preferably a region that hybridizes to the second oligonucleotide molecule but not to the target RNA. The base sequence of such second region can be appropriately determined according to the base sequence of the target RNA. The base sequence of the second region may also affect the efficiency of the subsequent amplification of cDNA, so that it is preferable to determine the base sequence in consideration of Tm value, GC content, and the like.

The first oligonucleotide molecule itself can be prepared by oligonucleotide synthesis methods known in the art. The first oligonucleotide molecule may contain conventionally known artificial nucleic acids such as bridged nucleic acid (BNA), phosphorothioate (PS)-oligo, peptide nucleic acid (PNA), morpholine oligos and 2′-O-substituted RNAs as long as extension of the 3′ end by the reverse transcription reaction is possible.

The second oligonucleotide molecule is an oligonucleotide molecule that hybridizes to a whole or part of the second region of the first oligonucleotide molecule. In the present embodiment, it is preferred that the second oligonucleotide molecule does not hybridize to the first region of the first oligonucleotide molecule. Although the synthesis method of the present embodiment is not limited by theory or action mechanism, it is presumed that the second oligonucleotide molecule hybridizes to the second region of the first oligonucleotide molecule, thereby playing a role in preventing unexpected hybridization and nonspecific binding of the polynucleotide to the second region.

The nucleotide length of the second oligonucleotide molecule may be the same length as the second region of the first oligonucleotide molecule or may be shorter than the second region. For example, the second oligonucleotide molecule has a nucleotide length of 35% or more and 100% or less, preferably 35% or more and 95% or less, and more preferably 35% or more and 90% or less of the nucleotide length of the second region of the first oligonucleotide molecule.

When the second oligonucleotide molecule has a nucleotide length shorter than the second region of the first oligonucleotide molecule, a plurality of second oligonucleotide molecules, preferably two or three second oligonucleotide molecules may be used. Each second oligonucleotide molecule hybridizes to different portions in the second region. The portions in the second region to which each second oligonucleotide molecule hybridizes may be adjacent or separated.

The base sequence of the second oligonucleotide molecule can be appropriately determined according to the base sequence of the second region of the first oligonucleotide molecule. The base sequence of the second oligonucleotide molecule may be a base sequence fully complementary to a whole or part of the second region of the first oligonucleotide molecule. Alternatively, the base sequence of the second oligonucleotide molecule may contain a base forming a mismatch site when hybridized with the second region of the first oligonucleotide molecule (hereinafter also referred to as “mismatched base”). For example, the ratio of the number of mismatched bases in the second oligonucleotide molecule (hereinafter also referred to as “mismatch rate”) is 50% or less, preferably 40% or less, and more preferably 12% or less. The mismatch rate is calculated by the following equation.

(Mismatch rate)={(Number of mismatched bases in second oligonucleotide molecule)/(Nucleotide length of second oligonucleotide molecule)}×100

When the second oligonucleotide molecule contains a mismatched base, the second oligonucleotide molecule has a nucleotide length of preferably 35% or more and more preferably 100% of the nucleotide length of the second region of the first oligonucleotide molecule.

The second oligonucleotide molecule may contain uracil in the base sequence. When the synthesis method of the present embodiment is performed using a second oligonucleotide molecule containing uracil, a part or whole of the second oligonucleotide molecule hybridized with the synthesized cDNA can be decomposed and removed by uracil DNA glycosylase (UNG).

The second oligonucleotide molecule itself can be prepared by oligonucleotide synthesis methods known in the art. The second oligonucleotide molecule may contain conventionally known artificial nucleic acids such as BNA, PS-oligo, PNA, morpholine oligo, and 2′-O-substituted RNA.

In the present embodiment, the 3′ end of the second oligonucleotide molecule may be modified so as not to extend. Examples of such modification include phosphorylation, biotinylation, and the like. The 3′ end can also be prevented from extending by introducing a modifying group such as didcoxyribonucleotide or an amino linker at the 3′ end of the second oligonucleotide molecule.

In the present embodiment, the second oligonucleotide molecule may be modified to improve stability of binding to the second region. Examples of such modification include introduction of a cap structure at 5′ end or 3′ end. Examples of the cap structure introduced at the 5′ end include a pyrene group, a trimethoxystilbene group, and the like. Examples of the cap structure introduced at the 3′ end include a 2′-(anthraquinon-2-yl-carboxamide)-2′-deoxyuridine group (hereinafter also referred to as “Uaq”) and the like. It is known that binding to the complementary strand is stabilized since the introduction of the cap structure improves the Tm value.

The reverse transcriptase is not particularly limited, and can be appropriately selected from known reverse transcriptases. Examples of the reverse transcriptase include avian myeloblastosis virus (AMV) reverse transcriptase, Moloney murine leukemia virus (MMLV) reverse transcriptase, and the like. An enzyme contained in a commercially available reverse transcription reaction kit may be used.

In the present embodiment, for example, the target RNA, the first oligonucleotide molecule, the second oligonucleotide molecule and the reverse transcriptase are mixed in water (preferably nuclease-free water), and the obtained mixture (hereinafter, also referred to as “composition for RT”) is placed under temperature conditions suitable for reverse transcription reactions, whereby cDNA is synthesized. The composition for RT may be further added with reagents used for general reverse transcription reaction such as buffer solutions (for example, Tris-HCl, etc.), dNTPs (mixtures of dATP, dCTP, dGTP and dTTP), inorganic salts (for example, NaCl, KCl, etc.), and RNase inhibitors. Such reagents are also included in commercially available reverse transcription reaction kits. The order in which the above components are mixed is not particularly limited. In the present embodiment, the first oligonucleotide molecule and the second oligonucleotide molecule may be previously mixed and used. However, it is not necessary to previously hybridize the first oligonucleotide molecule and the second oligonucleotide molecule. These oligonucleotide molecules spontaneously hybridize under temperature conditions suitable for reverse transcription reactions.

The addition amount of the first oligonucleotide molecule and the second oligonucleotide molecule is not particularly limited. The final concentrations of the first oligonucleotide molecule and the second oligonucleotide molecule in the composition for RT can be each appropriately determined from the range of 10 nM or more and 1000 nM or less, and preferably 10 nM or more and 250 nM or less. The final concentration of the second oligonucleotide molecule in the composition for RT may be the same as or different from the final concentration of the first oligonucleotide molecule.

The temperature conditions in the synthesis method of the present embodiment are not particularly different from the conditions for the normal reverse transcription reaction. Depending on the type of reverse transcriptase to be used, the temperature conditions can be appropriately selected from known temperature conditions for reverse transcription reaction. In the present embodiment, a reverse transcription reaction can be performed using a commercially available thermal cycler.

The second oligonucleotide molecule may be hybridized with the synthesized cDNA. The synthesis method of the present embodiment may include the step of removing at least a portion of the second oligonucleotide molecule after the synthesis of cDNA. For example, when the composition for RT after reverse transcription reaction is heated at 90° C. to 99° C., the second oligonucleotide molecule is dissociated from cDNA by heat denaturation. When the second oligonucleotide molecule contains uracil, at least a portion of the second oligonucleotide molecule can be decomposed by UNG after the synthesis of cDNA. For example, uracil of the second oligonucleotide molecule hybridized with cDNA is removed by adding UNG to the composition for RT after reverse transcription reaction and incubating at a predetermined temperature (for example, 25° C.). Since a portion that lacks uracil is structurally unstable, the second oligonucleotide molecule is cleaved at the portion that lacks uracil when the composition for RT is heated at 90° C. to 99° C. This decomposes a part or whole of the second oligonucleotide molecule.

In the present embodiment, in the same reaction system, cDNA may be synthesized using two or more types of target RNAs having different base sequences as a template. In this case, multiplex reverse transcription reaction can be performed by using a plurality of first oligonucleotide molecules having a first region according to the base sequence of each target RNA. The base sequence of the second region may be the same among the plurality of first oligonucleotide molecules or may be different from each other. The second oligonucleotide molecule can be appropriately designed according to the base sequence of the second region of the first oligonucleotide molecule.

The synthesis method of the present embodiment may further include the step of amplifying cDNA. Amplification of cDNA itself can be performed by a DNA amplification method known in the art such as PCR method, and real-time PCR method. Amplification of cDNA is performed, for example, by mixing cDNA, a forward primer that hybridizes to the cDNA, a reverse primer that hybridizes to a complementary strand of the cDNA, and a polymerase. Since the first oligonucleotide molecule can also hybridize to the complementary strand of cDNA, the first oligonucleotide molecule may be used in place of the reverse primer for amplification of cDNA.

The forward primer and the reverse primer can be appropriately designed based on the base sequence of cDNA, i.e., the base sequences of the target RNA and the second region of the first oligonucleotide molecule. The nucleotide length of each primer is usually 5 or more and 50 or less, and preferably 10 or more and 40 or less. The primers themselves can be prepared by oligonucleotide synthesis methods known in the art.

The addition amount of the forward primer and the reverse primer is not particularly limited. The final concentrations of the forward primer and the reverse primer in a composition for PCR can be each appropriately determined from the range of 0.1 μM or more and 1.5 μM or less, and preferably 0.35 μM or more and 1.5 μM or less. The final concentration of the reverse primer in the composition for PCR may be the same as or different from the final concentration of the forward primer.

The forward primer and the reverse primer may contain conventionally known artificial nucleic acids such as BNA, PS-oligo, PNA, morpholine oligo, and 2′-O-substituted RNA. The forward primer and the reverse primer may be labeled with a known labeling substance. Examples of the labeling substance include radioactive isotopes (for example, ³²P, ³⁵S, ³H, ¹⁴C, etc.), fluorescent dyes (for example, FITC, Texas Red (trademark), Alexa Fluor (trademark), 6-FAM, TAMRA, etc.), biotin, digoxigenin, and the like.

The polymerase is not particularly limited as long as it is a polymerase suitable for DNA amplification. Such a polymerase can be appropriately selected from known heat resistant DNA polymerases, and examples include Taq, Pfu, Tth, KOD, and the like. The polymerase contained in a commercially available PCR kit may be used.

In the present embodiment, cDNA, a forward primer, a reverse primer and/or a first oligonucleotide molecule and a polymerase are mixed in water, and the obtained mixture (hereinafter, also referred to as “composition for PCR”) is placed under temperature conditions suitable for DNA amplification, whereby double-stranded DNA is obtained using cDNA as a template. The composition for PCR may be further added with reagents used for general DNA amplification reactions such as buffer solutions (for example, Tris-HCl, etc.), dNTPs, and inorganic salts (for example, NaCl, KCl, etc.). Such reagents are also included in commercially available PCR kits and the like. The order of mixing each of the above components is not particularly limited. In the present embodiment, the forward primer and the reverse primer and/or the first oligonucleotide molecule may be previously mixed and used.

When cDNA is amplified by real-time PCR, the composition for PCR may be further added with a fluorescent intercalator (for example, SYBR (trademark) Green, etc.), and a fluorescently labeled probe modified with a fluorescent substance at 5′ end and with a quencher substance (for example, TaqMan (trademark) probe, etc.) at 3′ end.

The temperature conditions for amplification of cDNA are not particularly different from the conditions for normal PCR method or real-time PCR method. Depending on the type of polymerase to be used, the Tm value of the primer and the like, the temperature conditions can be appropriately selected from known temperature conditions for DNA amplification reaction. In the present embodiment, amplification of cDNA can be performed using a commercially available thermal cycler or real-time PCR device.

In the present embodiment, the composition for RT after reverse transcription reaction may be used as it is as a template cDNA. While the cDNA in the composition may hybridize to the second oligonucleotide molecule, the second oligonucleotide molecule is dissociated from cDNA by a heat denaturation step in DNA amplification. When the second oligonucleotide molecule contains uracil, a part or whole of the second oligonucleotide molecule hybridized with cDNA can be decomposed and removed by adding UNG to the composition for RT after reverse transcription reaction and incubating at a predetermined temperature (for example, 25° C.) and then performing DNA amplification.

In the present embodiment, the cDNA synthesis step and the cDNA amplification step may be performed in the same reaction system. In this case, for example, the target RNA, the first oligonucleotide molecule, the second oligonucleotide molecule, the reverse transcriptase, the forward primer, the reverse primer, and the polymerase are mixed. The obtained mixture is first placed under temperature conditions suitable for reverse transcription reactions to perform cDNA synthesis. Subsequently, the mixture is placed under temperature conditions suitable for a DNA amplification reaction to perform cDNA amplification. It is not necessary to transfer the mixture to another container between the reverse transcription reaction and the cDNA amplification reaction. Thus, the reverse transcription reaction and the cDNA amplification reaction can be performed in the same reaction system. In place of the reverse transcriptase and the polymerase, an enzyme having both reverse transcription activity and DNA polymerase activity (for example, Tth, etc.) may be used. The above mixture may be further added with reagents used for general reverse transcription reaction and DNA amplification reaction such as buffer solutions (for example, Tris-HCl, etc.), dNTPs, inorganic salts (for example, NaCl, KCl, etc.), and RNase inhibitors.

### [2. Method for Detecting Target RNA]

The scope of the present disclosure also includes a method for detecting target RNA (hereinafter also referred to as “detection method”). In the detection method of the present embodiment, first, a target RNA, a first oligonucleotide molecule, a second oligonucleotide molecule, and a reverse transcriptase are mixed, and cDNA is synthesized using the target RNA as a template. Details of the components used for cDNA synthesis (reverse transcription reaction), conditions for the reverse transcription reaction and the like are the same as those described for the synthesis method of the present embodiment described above.

Next, in the detection method of the embodiment, the synthesized cDNA is amplified, and an amplification product of the cDNA is detected. Details of the components used for cDNA amplification, conditions for the amplification reaction and the like are the same as those described for the synthesis method of the present embodiment described above. As used herein, the term “detecting” includes qualitatively determining the presence or absence of an amplification product, quantifying the amplification product, and detecting semiquantitatively the abundance of the amplification product. Semi-quantitative detection refers that the abundance of the amplification product is indicated stepwise like “−”, “+”, “++” (negative, weak positive, strong positive), and the like. For example, when a result that an amplification product has been detected is obtained, the result indicates the presence of target RNA.

A means for detecting an amplification product is not particularly limited, and can be appropriately selected from known methods. For example, an amplification product may be detected by electrophoresis. Specifically, a reaction solution after the amplification reaction is electrophoresed on an agarose gel containing ethidium bromide to confirm the presence or absence of the amplification product, and the amount of the amplification product, if any. The amplification product may be detected by acquiring optical information such as fluorescence intensity, turbidity, and absorbance, from the reaction solution after the amplification reaction. For example, the amplification product may be detected by measuring the fluorescence intensity by an intercalator method using a fluorescent substance capable of binding to double-stranded DNA such as SYBR (trademark) Green.

Alternatively, the amplification product may be detected by detecting fluorescence generated from the probe by a method using the fluorescently labeled probe (for example, TaqMan (trademark) probe, etc.) modified with a fluorescent substance at the 5′ end and with a quencher substance at the 3′ end. Such a probe is designed to hybridize to a region different from the region to which the above forward primer and/or reverse primer hybridize in the amplification product to be detected. The final concentration of the fluorescently labeled probe in the composition for PCR is not particularly limited, and can be appropriately determined, for example, from the range of 0.1 μM or more and 5 μM or less, and preferably 0.2 μM or more and 0.8 μM or less.

### [3. Reagent Kit]

The scope of the present disclosure also includes a reagent kit. This reagent kit can be used to perform the synthesis method of the present embodiment and the detection method of the present embodiment. The reagent kit of the present embodiment includes a first oligonucleotide molecule that hybridizes to a target RNA, and a second oligonucleotide molecule that hybridizes to the first oligonucleotide molecule. Details of the first oligonucleotide molecule and the second oligonucleotide molecule are the same as those described for the synthesis method of the present embodiment described above.

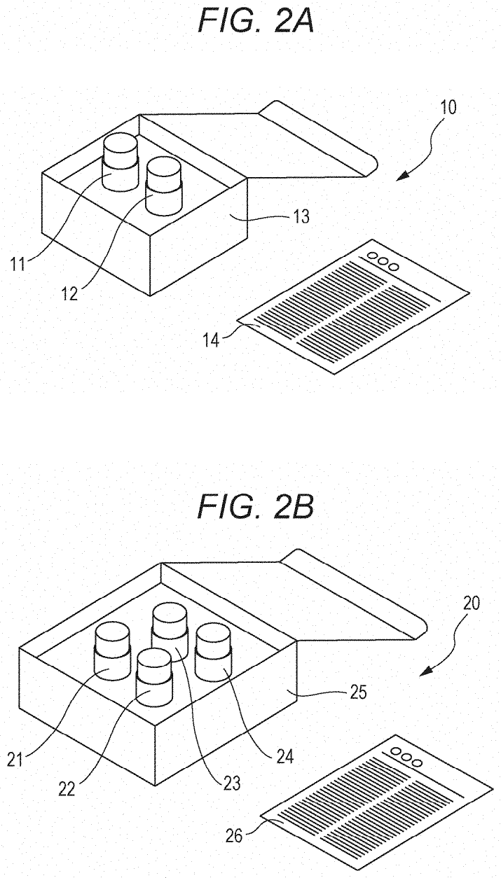

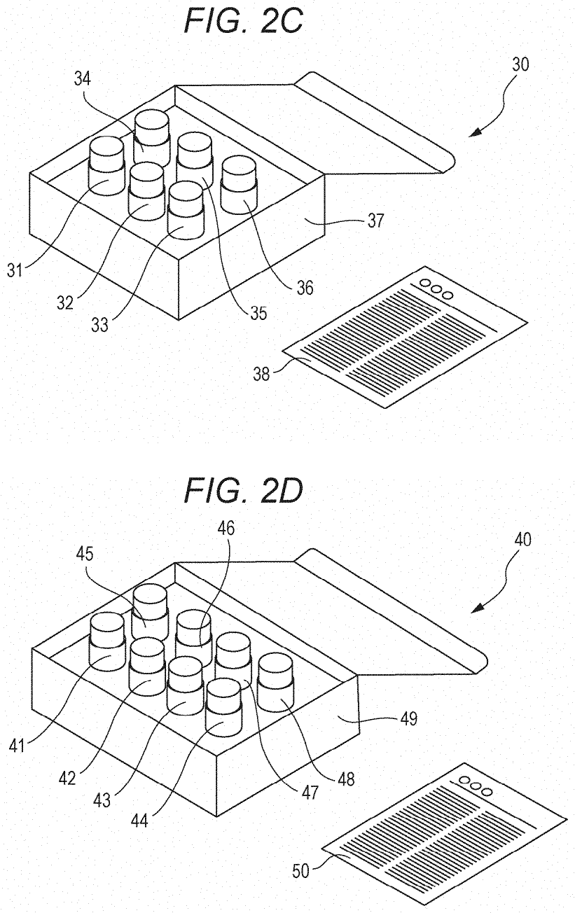

Containers respectively containing the first oligonucleotide molecule and the second oligonucleotide molecule may be contained in a box and provided to the user. The box may include all the above containers, or may contain some containers. The box may include an attached document describing a method of using the first oligonucleotide molecule and the second oligonucleotide molecule and the like. FIG. 2A shows an example of the reagent kit of the present embodiment. In the figure, 10 denotes a reagent kit, 11 denotes a first container containing a first oligonucleotide molecule, 12 denotes a second container containing a second oligonucleotide molecule, 13 denotes a packing box, and 14 denotes the attached document.

The concentration of the first oligonucleotide molecule in the first container may be a concentration that can be adjusted to the above final concentration when used for preparation of the composition for RT. Therefore, the concentration of the first oligonucleotide molecule in the container can be appropriately determined from a volume ratio of a total amount of the composition for RT and an addition amount of the first oligonucleotide molecule. In the present embodiment, the concentration of the first oligonucleotide molecule in the first container is, for example, 0.05 μM or more and 100 μM or less, and preferably 0.25 μM or more and 10 μM or less.

The concentration of the second oligonucleotide molecule in the second container can also be determined similar to the concentration of the first oligonucleotide molecule. In the present embodiment, the concentration of the second oligonucleotide molecule in the second container is, for example, 0.05 μM or more and 100 μM or less, and preferably 0.25 μM or more and 10 μM or less. The concentration of the second oligonucleotide molecule in the second container may be the same as or different from the concentration of the first oligonucleotide molecule in the first container.

The reagent kit of the present embodiment may further include a reverse transcriptase. The reagent kit of the present embodiment may further include dNTP. The reverse transcriptase itself is known in the art, and can be appropriately selected from, for example, enzymes derived from retrovirus such as AMV and MMLV. The dNTP may be a mixture of dATP, dCTP, dGTP and dTTP. FIG. 2B shows an example of a reagent kit further including a reverse transcriptase and dNTP. In the figure, 20 denotes a reagent kit, 21 denotes a first container containing a first oligonucleotide molecule, 22 denotes a second container containing a second oligonucleotide molecule, 23 denotes a third container containing a reverse transcriptase, 24 denotes a fourth container containing dNTP, 25 denotes a packing box, and 26 denotes an attached document. Although not shown, the reagent kit of the present embodiment may further include a buffer solution suitable for reverse transcription reactions.

The reagent kit of the present embodiment may further includes a forward primer that hybridizes to cDNA synthesized by extension of the first oligonucleotide molecule, and a reverse primer that hybridizes to a complementary strand of the cDNA. Details of the forward primer and the reverse primer are the same as those described for the synthesis method of the present embodiment described above. FIG. 2C shows an example of a reagent kit further including a forward primer and a reverse primer. In the figure, 30 denotes a reagent kit, 31 denotes a first container containing a first oligonucleotide molecule, 32 denotes a second container containing a second oligonucleotide molecule, 33 denotes a third container containing a reverse transcriptase, 34 denotes a fourth container containing dNTP, 35 denotes a fifth container containing a forward primer, 36 denotes a sixth container containing a reverse primer, 37 denotes a packing box, and 38 denotes an attached document. Although not shown, the reagent kit of the present embodiment may further include a buffer solution suitable for reverse transcription reactions.

The concentration of the forward primer in the fifth container may be any concentration that can be adjusted to the above final concentration when used for preparation of the composition for PCR. Therefore, the concentration of the forward primer in the container can be appropriately determined from a volume ratio of the total amount of the composition for PCR and an addition amount of the forward primer. In the present embodiment, the concentration of the forward primer in the fifth container is, for example, 6 μM or more and 100 μM or less, and preferably 15 μM or more and 100 μM or less.

The concentration of the reverse primer in the sixth container can also be determined similar to the concentration of the forward primer. In the present embodiment, the concentration of the reverse primer in the sixth container is, for example, 3 μM or more and 100 μM or less, and preferably 15 μM or more and 100 μM or less. The concentration of the reverse primer in the sixth container may be the same as or different from the concentration of the forward primer in the fifth container.

The reagent kit of the present embodiment may further include a polymerase. The reagent kit of the present embodiment may further include a fluorescently labeled probe. Details of the polymerase and the fluorescently labeled probe are the same as those described for the synthesis method and detection method of the present embodiment described above. FIG. 2D shows an example of a reagent kit further including a polymerase and a fluorescently labeled probe. In the figure, 40 denotes a reagent kit, 41 denotes a first container containing a first oligonucleotide molecule, 42 denotes a second container containing a second oligonucleotide molecule, 43 denotes a third container containing a reverse transcriptase, 44 denotes a fourth container containing dNTP, 45 denotes a fifth container containing a forward primer, 46 denotes a sixth container containing a reverse primer, 47 denotes a seventh container containing a polymerase, 48 denotes an eighth container containing a fluorescently labeled probe, 49 denotes a packing box, and 50 denotes an attached document. Although not shown, the reagent kit of the present embodiment may further include a buffer solution suitable for reverse transcription reactions and a buffer solution suitable for DNA amplification reactions.

The concentration of the fluorescently labeled probe in the eighth container may be any concentration that can be adjusted to the above final concentration when used for the preparation of the composition for PCR. Therefore, the concentration of the fluorescently labeled probe in the container can be appropriately determined from a volume ratio of the total amount of the composition for PCR and an addition amount of the fluorescently labeled probe. In the present embodiment, the concentration of the fluorescently labeled probe in the eighth container is, for example, 2 μM or more and 25 μM or less, and preferably 2 μM or more and 10 μM or less.

In the present embodiment, at least two of the first oligonucleotide, the second oligonucleotide, the forward primer and the reverse primer may be contained in the same container. Preferably, the first oligonucleotide and the second oligonucleotide are contained in the same container. Alternatively, the forward primer and the reverse primer are contained in the same container. More preferably, the first oligonucleotide and the second oligonucleotide are contained in the same container, and the forward primer and the reverse primer are contained in another same container.

Hereinbelow, the present invention will be described in detail by examples, but the present invention is not limited to these examples.

### EXAMPLES

### Example 1

In Example 1, cDNA was synthesized from target RNA (miRNA) by the method of EP 1 851 336 B, the method of U.S. Pat. No. 7,575,863, and the synthesis method of the present embodiment, and the cDNA was amplified and detected by real-time PCR. Then, the lower limit of detection and the lower limit of quantification of the amplification product were compared. In Example 1, the experiment was performed in triplicate.

(1) Synthesis of cDNA

### (1.1) Target RNA and Reverse Transcription Primer

An oligonucleotide of a base sequence represented by SEQ ID NO: 1 was used as target RNA. This base sequence is a base sequence of miR302a. An oligonucleotide of a base sequence represented by SEQ ID NO: 2 was used as a reverse transcription primer (hereinafter, referred to as “RT primer”). This oligonucleotide is not only an RT primer used in the method of EP 1 851 336 B, but also a first oligonucleotide molecule used in the synthesis method of the present embodiment. In the base sequence represented by SEQ ID NO: 2, 1st to 24th base sequence counted from 5′ side is a region (second region) which does not hybridize to the target RNA, and 25th to 34th base sequence is a region (first region) that hybridizes to the target RNA. An oligonucleotide of a base sequence represented by SEQ ID NO: 3 was used as a stem-loop RT primer used in the method of U.S. Pat. No. 7,575,863. An oligonucleotide of a base sequence represented by SEQ ID NO: 4 was used as a second oligonucleotide molecule used in the synthesis method of the present embodiment. This base sequence is fully complementary to the 1st to 24th base sequences counted from the 5′ side of the base sequence represented by SEQ ID NO: 2. The base sequences of the oligonucleotides are shown below. Underlined portions indicate regions where the target RNA hybridizes to each RT primer.

Target RNA (miR302a):
(SEQ ID NO: 1)
5′-UAAGUGCUUCCAUGUUUUGGUGA-3′
RT Primer:
(SEQ ID NO: 2)
5′-CGAGGTATTCGCACTGGATACGACTCACCAAAAC-3′
Stem-loop RT primer:
(SEQ ID NO: 3)
5′-GTCGTATCCAGTGCAGGGTCCGAGGTATTCGCACTGGATACGACTCA
CCAAAAC-3′
Second oligonucleotide molecule:
(SEQ ID NO: 4)
5′-GTCGTATCCAGTGCGAATACCTCG-3′

### (1.2) Reverse Transcription Reaction

A mixture of the following composition was prepared using the above oligonucleotide and TaqMan (trademark) MicroRNA Reverse Transcription Kit (Applied Biosystems, product number 4366597). The kit contained 100 mM dNTP, 10×buffer, an RNase inhibitor and a reverse transcriptase. In the synthesis method of the present embodiment, the RT primer of SEQ ID NO: 2 and the second oligonucleotide molecule were previously mixed to prepare a 250 nM RT primer. The concentration of target RNA was adjusted with nuclease-free water to obtain solutions containing 500, 10³, 10⁴, 10⁵, 10⁶ or 10⁷ copies of miRNA per pt. In the synthesis method of the present embodiment and the method of EP 1 851 336 B, solutions containing 10³, 10⁵ or 10⁷ copies of miRNA per μL were used as target RNA. In the method of U.S. Pat. No. 7,575,863, solutions containing 500, 10³, 10⁴, 10⁵, or 10⁶ copies of miRNA per μL were used as target RNA. A mixture was similarly prepared except that yeast tRNA was used in place of the target RNA to obtain a negative control.

### <Composition Per Well>

| | |
|---|---|
| Nuclease-free water | 8.16 μL |
| 100 mM dNTP | 0.15 μL |
| 10 × Buffer | 1.5 μL |
| RNase Inhibitor | 0.19 μL |
| Reverse transcriptase | 1 μL |
| 250 nM RT primer | 3 μL |
| Target RNA | 1 μL |
| Total | 15 μL |

The prepared mixture was set to GeneAmp PCR System 9700 (Applied Biosystems), and reverse transcription reaction was performed under temperature conditions of 16° C. for 30 minutes, 42° C. for 30 minutes, and 85° C. for 5 minutes.

(2) Amplification and Detection of cDNA by Real-Time PCR

### (2.1) Primer Set and Probe for PCR

Oligonucleotides of base sequences represented by SEQ ID NOS: 5, 6 and 7 were used as a forward primer, a reverse primer, and a probe, respectively. The base sequences of the oligonucleotides are shown below. The forward primer and the reverse primer were mixed to prepare a primer mix. The concentration of the forward primer in the primer mix was 30 μM, and the concentration of the reverse primer was 15 μM. The probe was a TaqMan (trademark) probe modified with a fluorescent substance at 5′ end and with a quencher substance at 3′ end.

Forward primer:
(SEQ ID NO: 5)
5′-TAAGTGCTTCCATGTTT-3′
Reverse primer:
(SEQ ID NO: 6)
5′-CGAGGTATTCGCA-3′
TaqMan (trademark) Probe:
(SEQ ID NO: 7)
5′-ATACGACTCACCA-3′

### (2.2) Real-Time PCR

A mixture of the following composition was prepared using the above reverse transcription product, the primer mix and the probe, and TaqMan (trademark) Universal Master Mix II, with UNG (Applied Biosystems, product number 4440038).

### <Composition Per Well>

| | |
|---|---|
| Primer mix | 1 μL |
| 8 μM Probe | 2 μL |
| Master mix II | 10 μL |
| Reverse transcript | 1.33 μL |
| Distilled water | 5.67 μL |
| Total | 20 μL |

The prepared mixture was set to 7500 Fast Real-Time PCR System (Applied Biosystems), and PCR was performed under the following temperature conditions. Heating and cooling were performed at a rate of 1.6° C./sec.

### <Temperature Conditions>

At 95° C. for 10 minutes,
40 cycles at 95° C. for 15 seconds and at 55° C. for 60 seconds.

### (3) Analysis and Results

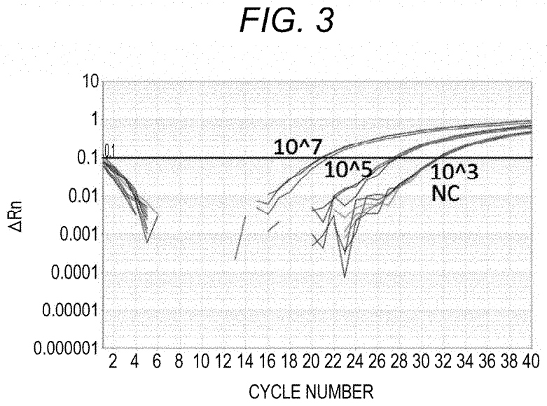

The amplification plots obtained by the method of EP 1 851 336 B, the method of U.S. Pat. No. 7,575,863, and the synthesis method of the present embodiment are shown in FIGS. 3 to 5, respectively. In these plots, the abscissa represents the cycle number, and the ordinate represents the change in fluorescence intensity (ΔRn). In the figures, “10\hat{ }3”, “10\hat{ }5”, “10\hat{ }6” and “10\hat{ }7” represent cDNA amplification products derived from specimens containing 10³, 10⁵, 10⁶ and 10⁷ copies of the target RNA, respectively. “NC” indicates a negative control. In the amplification plots obtained by the methods of EP 1 851 336 B and U.S. Pat. No. 7,575,863, a threshold value of ΔRn was set to 0.1, and in the amplification plot obtained by the synthesis method of the present embodiment, a threshold value of ΔRn was set to 0.03. Then, Ct (Threshold Cycle) values were obtained from the points of contact with the respective amplification curves. The copy number of the target RNA in the mixture of (1.2) above was defined as an initial template amount, and a calibration curve was created from the Ct value and a logarithm of the initial template amount (not shown).

The lower limit of detection and the lower limit of quantification of each method were determined based on the calibration curve. The results are shown in Table 1. Lower limit of quantification (1) was determined from the range in which the value of correlation coefficient (R²) of the calibration curve was larger than 0.98. Lower limit of quantification (2) was determined from the range in which the slope of the calibration curve was −3.91 or more and −2.92 or less (within amplification efficiency ±20%). The lower limit of detection is a value at which a p value is smaller than 0.01 when T test (two-sided test) is performed on the Ct value of the negative control (yeast tRNA). As the T test, Excel (trademark) function T. TEST was used.

TABLE 1

| | Lower limit of | Lower limit of | |
|---|---|---|---|
| | quantification | quantification | Lower limit |
| | (1) | (2) | of detection |
| EP 1 851 336 B | 10⁵ | 10⁵ | 10⁵ |
| U.S. Pat. No. 7,575,863 | 10³ | 10⁵ | 10³ |
| Present embodiment | 10³ | 10³ | 10³ |

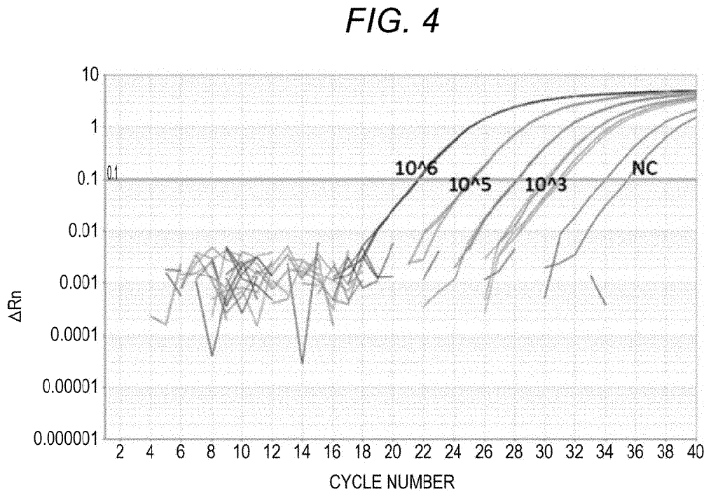

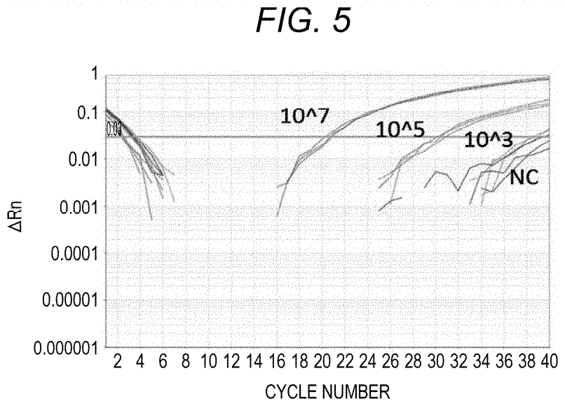

As shown in Table 1, the synthesis method of the present embodiment was found to have higher performance in detection and quantification of the amplification products than the methods of the prior art EP 1 851 336 B and U.S. Pat. No. 7,575,863. As shown in FIG. 4, in the method of U.S. Pat. No. 7,575,863, the negative control was also amplified, and nonspecific amplification was observed. On the other hand, as shown in FIG. 5, almost no nonspecific amplification was observed in the synthesis method of the present embodiment. Accordingly, it was shown that the synthesis method of the present embodiment has higher sensitivity and specificity in synthesis and amplification of cDNA from small RNAs, as compared to the prior art.

### Example 2

In Example 2, cDNA was synthesized from target RNA by the synthesis method of the present embodiment using second oligonucleotide molecules of different nucleotide lengths, and the cDNA was amplified and detected by real-time PCR. Then, the lower limit of detection and the lower limit of quantification of the amplification product were compared. In Example 2, the experiment was performed in triplicate.

### (1) Target RNA and Reverse Transcription Primer

The oligonucleotides of the base sequences represented by SEQ ID NOS: 1 and 2 were used as target RNA and RT primers, respectively. Oligonucleotides of the base sequences shown below were used as the second oligonucleotide molecules. Hereinafter, these second oligonucleotide molecules are also referred to as “Lid”, “Lid-S1”, “Lid-S2” and “Lid-S3”, respectively. The base sequences of each second oligonucleotide molecule are shown below. The ratios of the nucleotide length of each second oligonucleotide molecule to the nucleotide length (24 bases) of the second region of the RT primer were shown in Table 2.

Lid:
(SEQ ID NO: 4)
5′-GTCGTATCCAGTGCGAATACCTCG-3′
Lid-S1:
(SEQ ID NO: 8)
5′-GTCGTATCCAGTGCGAATACC-3′
Lid-S2:
(SEQ ID NO: 9)
5′-GTCGTATCCAGTGCGAAT-3′
Lid-S3:
5′-GTCGTATCC-3′

TABLE 2

| | Nucleotide | Ratio (%) to nucleotide |
|---|---|---|
| | length | length of second region |
| Lid | 24 | 100 |
| Lid-S1 | 21 | 87.5 |
| Lid-S2 | 18 | 75.0 |
| Lid-S3 | 9 | 37.5 |

(2) cDNA Synthesis, Amplification and Detection

Reverse transcription reaction was performed to synthesize cDNA in the same manner as in Example 1 except that the above second oligonucleotide molecule was used. The obtained cDNA was amplified and detected by real-time PCR in the same manner as in Example 1 to obtain an amplification plot (not shown).

### (3) Analysis and Results

A calibration curve was created from each amplification plot, with a threshold value of ΔRn of 0.05. The lower limit of detection and the lower limit of quantification were determined in the same manner as in Example 1 based on the obtained calibration curve. The results are shown in Table 3.

TABLE 3

| | Lower limit of | Lower limit of | |
|---|---|---|---|
| | quantification | quantification | Lower limit of |
| | (1) | (2) | detection |
| Lid | 10³ | 10³ | 10³ |
| Lid-S1 | 10³ | 10³ | 10³ |
| Lid-S2 | 10³ | 10³ | 10³ |
| Lid-S3 | 10³ | 10³ | 10³ |

As shown in Table 3, it was found that performance of detection and quantification of the amplification products was high even using a second oligonucleotide molecule of any nucleotide length. From the amplification plot, almost no nonspecific amplification was observed even using a second oligonucleotide molecule of any nucleotide length.

### Example 3

In Example 3, cDNA was synthesized from target RNA by the synthesis method of the present embodiment using a second oligonucleotide molecule that forms a mismatch site when hybridized with the second region of the RT primer, and the cDNA was amplified and detected by real-time PCR. Then, the lower limit of detection and the lower limit of quantification of the amplification product were compared. In Example 3, the experiment was performed in triplicate.

### (1) Target RNA and Reverse Transcription Primer

The oligonucleotides of the base sequences represented by SEQ ID NOS: 1 and 2 were used as target RNA and RT primers, respectively. Oligonucleotides of base sequences represented by SEQ ID NOS: 4 and 10 to 13 were used as the second oligonucleotide molecules. The oligonucleotides of the base sequences represented by SEQ ID NOS: 10 to 13 are also referred to as “Lid-M1”, “Lid-M2”, “Lid-M3” and “Lid-M4”, respectively. The base sequences of each second oligonucleotide molecule are shown below. Underlined portions indicate mismatched bases in the second oligonucleotide molecules. The mismatch rate of the second oligonucleotide molecule was shown in Table 4.

Lid:
(SEQ ID NO: 4)
5′-GTCGTATCCAGTGCGAATACCTCG-3′
Lid-M1:
(SEQ ID NO: 10)
5′-GTCGTATCCATTGCTAATACCTCG-3′
Lid-M2:
(SEQ ID NO: 11)
5′-GTCGTATCCATTACTAATACCTCG-3′
Lid-M3:
(SEQ ID NO: 12)
5′-GTTGTGTTCGGTGTGGATGCTTTG-3′
Lid-M4:
(SEQ ID NO: 13)
5′-GTTGTGTTTGGTGTGGGTGTTTTG-3′

TABLE 4

| | Number of | |
|---|---|---|
| | mismatched bases | Mismatch rate (%) |
| Lid | 0 | 0 |
| Lid-M1 | 2 | 8.3 |
| Lid-M2 | 3 | 12.5 |
| Lid-M3 | 9 | 37.5 |
| Lid-M4 | 12 | 50.0 |

(2) cDNA Synthesis, Amplification and Detection

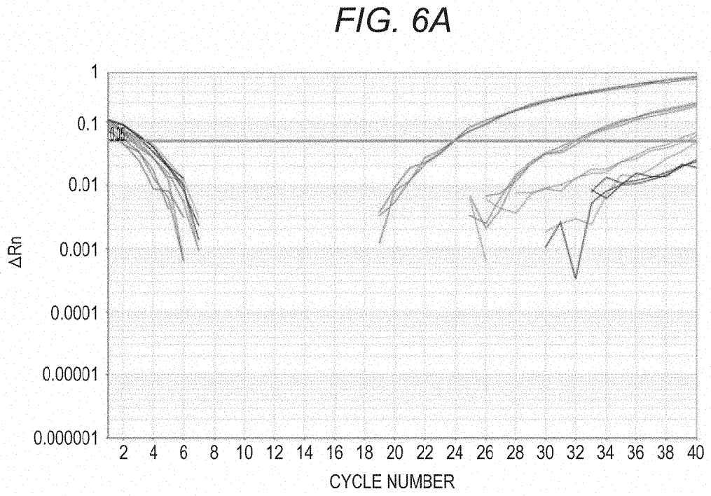

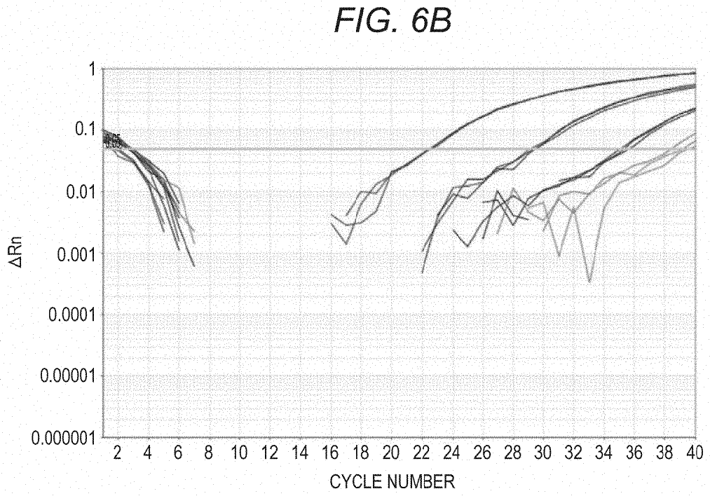

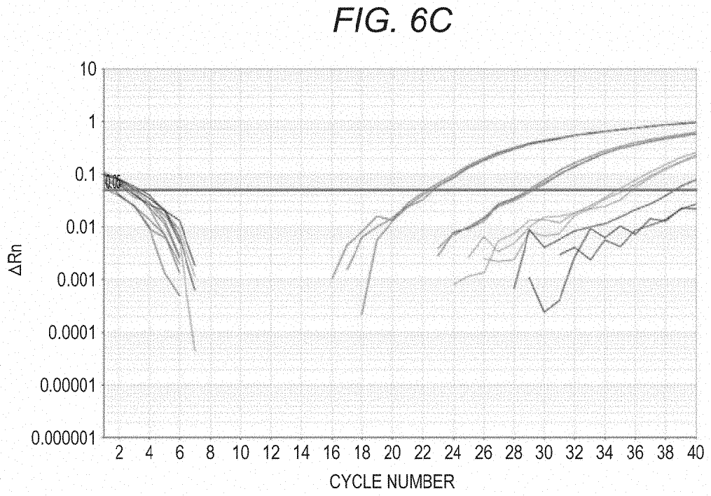

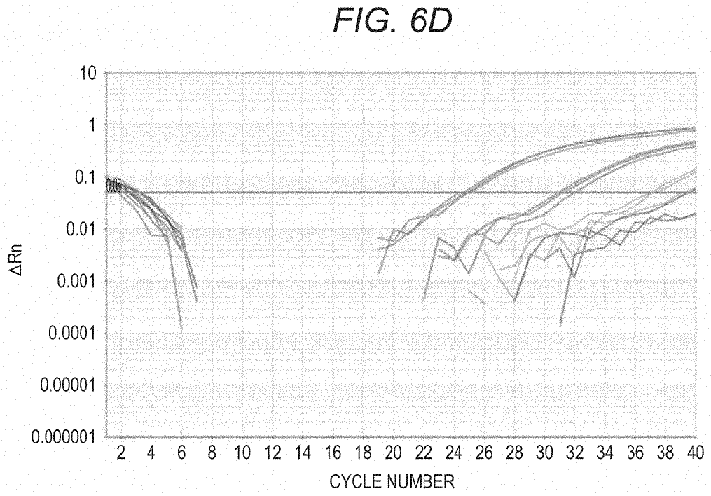

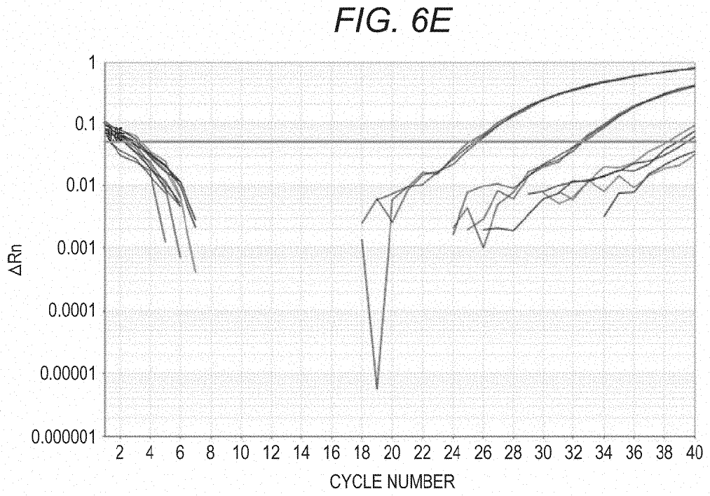

Reverse transcription reaction was performed to synthesize cDNA in the same manner as in Example 1 except that the above second oligonucleotide molecule was used. As in Example 1, the obtained cDNA was amplified and detected by real-time PCR. The obtained amplification plots are shown in FIGS. 6A to 6E.

### (3) Analysis and Results

A calibration curve was created from each amplification plot, with a threshold value of ΔRn of 0.05. The lower limit of detection and the lower limit of quantification were determined in the same manner as in Example 1 based on the obtained calibration curve. The results are shown in Table 5.

TABLE 5

| | Lower limit of | Lower limit of | |
|---|---|---|---|
| | quantification | quantification | Lower limit of |
| | (1) | (2) | detection |
| Lid | 10³ | 10³ | 10³ |
| Lid-M1 | 10³ | 10³ | 10³ |
| Lid-M2 | 10³ | 10³ | 10³ |
| Lid-M3 | 10³ | 10³ | 10³ |
| Lid-M4 | 10³ | 10³ | 10³ |

As shown in Table 5, it was found that performance of detection and quantification of the amplification products was high even using the second oligonucleotide molecule having a mismatched base. From FIG. 6A to 6E, almost no nonspecific amplification was observed even when about 50% of mismatch was present between the second region of the RT primer and the second oligonucleotide molecule.

### Example 4

In Example 4, cDNA was synthesized from target RNA by the synthesis method of the present embodiment using a second oligonucleotide molecule having a CAP structure added at 3′ end, and the cDNA was amplified and detected by real-time PCR. Then, the lower limit of detection and the lower limit of quantification of the amplification product were compared. In Example 4, the experiment was performed in triplicate.

### (1) Target RNA and Reverse Transcription Primer

An oligonucleotide of a base sequence represented by SEQ ID NO: 1 was used as target RNA. The oligonucleotides of the base sequences represented by SEQ ID NOS: 2 and 14 were used as RT primers. Hereinafter, these RT primers are also referred to as “RT-primer 1” and “RT-primer 2”, respectively. Oligonucleotides of base sequences represented by SEQ ID NOS: 4 and 15 were used as the second oligonucleotide molecules. In Example 4, Uaq was added as a cap structure at 3′ ends of these oligonucleotides. The addition of 3′-Uaq CAP is expected to improve the Tm value and improve stability of binding to the complementary strand. Hereinafter, the second oligonucleotide molecules added with 3′-Uaq CAP are also referred to as “Lid-Cap1” and “Lid-Cap2”, respectively. A chemical structure of 3′-Uaq CAP is shown below. The base sequences of the RT primer and the second oligonucleotide molecule are shown below.

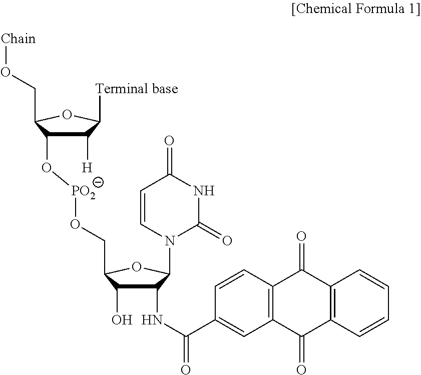

RT-primer 1:
(SEQ ID NO: 2)
5′-CGAGGTATTCGCACTGGATACGACTCACCAAAAC-3′
RT-primer 2:
(SEQ ID NO: 14)
5′-GTCCGAGGTATTCGCACTGGATACGACTCACCAAAAC-3′
Lid:
(SEQ ID NO: 4)
5′-GTCGTATCCAGTGCGAATACCTCG-3′
Lid-Cap 1:
(SEQ ID NO: 4)
5′-GTCGTATCCAGTGCGAATACCTCG-Uaq-3′
Lid-Cap 2:
(SEQ ID NO: 15)
5′-GTCGTATCCAGTGCGAATACCTCGGAC-Uaq-3′

(2) cDNA Synthesis, Amplification and Detection

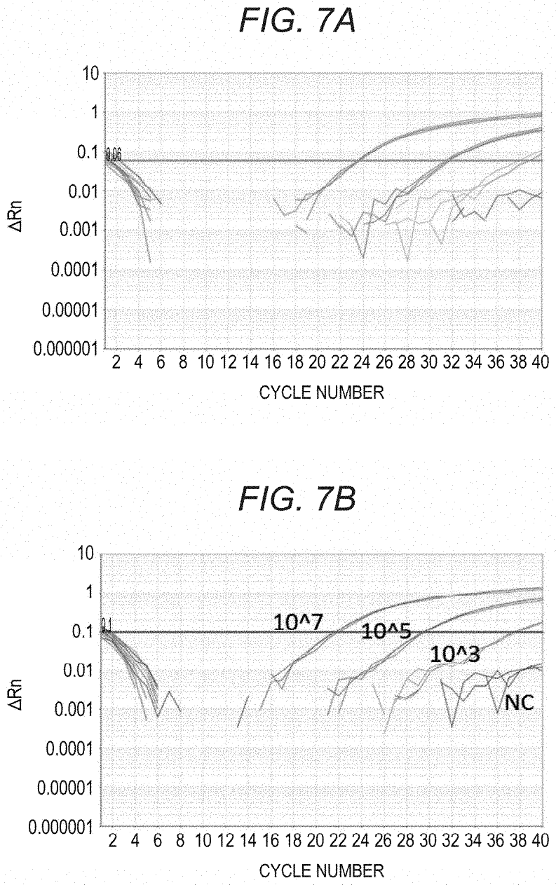

Reverse transcription reaction was performed to synthesize cDNA in the same manner as in Example 1 except that the RT primer and the second oligonucleotide molecule were used. Lid or Lid-Cap 1 was used as a second oligonucleotide molecule for RT-primer 1, and Lid-Cap 2 was used as a second oligonucleotide molecule for RT-primer 2. As in Example 1, the obtained cDNA was amplified and detected by real-time PCR. In amplification of cDNA synthesized using RT-primer 2, in place of the reverse primer of the base sequence represented by SEQ ID NO: 6, a reverse primer of a base sequence represented by SEQ ID NO: 16 below was used. The amplification plots using Lid-Cap 1 and Lid-Cap 2 are shown in FIGS. 7A and 7B, respectively.

Reverse primer:
(SEQ ID NO: 16)
5′-CCGAGGTATTCGCACTG-3′

### (3) Analysis and Results

A calibration curve was created from each amplification plot, with a threshold value of ΔRn of 0.1. The lower limit of detection and the lower limit of quantification were determined in the same manner as in Example 1 based on the obtained calibration curve. The results are shown in Table 6.

TABLE 6

| | Lower limit of | Lower limit of | |
|---|---|---|---|
| | quantification | quantification | Lower limit |
| | (1) | (2) | of detection |
| Lid | 10³ | 10³ | 10³ |
| Lid-Cap 1 | 10³ | 10³ | 10³ |
| Lid-Cap 2 | 10³ | 10³ | 10³ |

As shown in Table 6, it was found that performance of detection and quantification of the amplification products was high also when using the second oligonucleotide molecule having a CAP structure added at the 3′ end, as well as when using an unmodified second oligonucleotide molecule. FIGS. 7A and 7B show that nonspecific amplification is suppressed when using the second oligonucleotide molecule having a CAP structure added at the 3′ end. Accordingly, it was suggested that the addition of the CAP structure to the 3′ end of the second oligonucleotide molecule has an effect of suppressing nonspecific amplification.

What is claimed is:

1. A method for synthesizing cDNA by mixing a target RNA, a first oligonucleotide molecule, a second oligonucleotide molecule, and a reverse transcriptase, using the target RNA as a template, the first oligonucleotide molecule comprising a first region that hybridizes to the target RNA at 3′ end, and comprising a second region that hybridizes to the second oligonucleotide molecule on 5′ side of the first region.

2. The method according to claim 1, wherein
the first region of the first oligonucleotide molecule hybridizes to the target RNA, and the second region of the first oligonucleotide molecule hybridizes to the second oligonucleotide molecule, and
the reverse transcriptase extends the 3′ end of the first oligonucleotide molecule hybridized with the target RNA and the second oligonucleotide molecule to synthesize cDNA.

3. The method according to claim 1, wherein the target RNA is small RNA.

4. The method according to claim 3, wherein the small RNA is any one selected from the group consisting of miRNA, snRNA, snoRNA, piRNA, pri-miRNA, pre-miRNA, siRNA, and shRNA.

5. The method according to claim 1, wherein a nucleotide length of the first region of the first oligonucleotide molecule is 3 or more and 15 or less.

6. The method according to claim 1, wherein a nucleotide length of the second region of the first oligonucleotide molecule is 10 or more and 40 or less.

7. The method according to claim 1, wherein a nucleotide length of the second oligonucleotide molecule is 35% or more and 100% or less of the nucleotide length of the second region of the first oligonucleotide molecule.

8. The method according to claim 1, wherein a ratio of the number of bases forming a mismatch site when hybridized with the second region of the first oligonucleotide molecule in the second oligonucleotide molecule is within 50%.

9. The method according to claim 1, wherein the 3′ end of the second oligonucleotide molecule is modified so as not to extend.

10. The method according to claim 1, further comprising a step of amplifying the cDNA.

11. The method according to claim 10, wherein the cDNA amplification step comprises mixing the cDNA, a forward primer that hybridizes to the cDNA, a reverse primer that hybridizes to a complementary strand of the cDNA and/or a first oligonucleotide molecule, and a polymerase.

12. The method according to claim 10, wherein the cDNA amplification step is performed by real-time PCR.

13. The method according to claim 1, further comprising the step of removing at least a portion of the second oligonucleotide molecule after the synthesis of cDNA.

14. The method according to claim 1, wherein the second oligonucleotide molecule contains uracil.

15. The method according to claim 14, further comprising the step of decomposing at least a portion of the second oligonucleotide molecule with uracil N-glycosylase after the synthesis of cDNA.

16. The method according to claim 1, wherein the cDNA synthesis step and the amplification step are performed in the same reaction system.

17. The method according to claim 1, further comprising a step of amplifying the synthesized cDNA.

18. The method according to claim 17, wherein, the amplifying step is performed by real-time PCR to quantify the target RNA.

19. A method for synthesizing cDNA, comprising
contacting a template target RNA, a first oligonucleotide molecule, a second oligonucleotide molecule, and a reverse transcriptase, the first oligonucleotide molecule comprising a first region that hybridizes to the target RNA at 3′ end, and comprising a second region that hybridizes to the second oligonucleotide molecule on 5′ side of the first region, and
conducting extension reaction from the 3′ end of the first region of the first oligonucleotide molecule to synthesize cDNA.

20. A method for synthesizing cDNA, comprising
conducting a reverse transcription reaction of a target RNA in a sample with a first oligonucleotide molecule and a second oligonucleotide molecule to synthesize the cDNA, the first oligonucleotide molecule comprising a first region that hybridizes to the target RNA at 3′ end, and comprising a second region that hybridizes to the second oligonucleotide molecule on 5′ side of the first region, and
amplifying and detecting the cDNA.
